(19) United States
(12) Patent Application Publication
(10) Pub. No.: US 2007/0027428 A1
(43) Pub. Date: Feb. 1, 2007

(54) Vial system and method for needle-less injector

(75) Inventor: John William Bingham, Elizabeth, CO (US)

Correspondence Address: NIXON PEABODY, LLP, 401 9TH STREET, NW, SUITE 900, WASHINGTON, DC, 20004-2128, US

(73) Assignee: PharmaJet, Inc., Goldon, CO (US)

(21) Appl. No.: 11/453,248

(22) Filed: Jun. 15, 2006

Related U.S. Application Data
(63) Continuation-in-part of application No. 11/121,439, filed on May 3, 2005.
(63) Continuation-in-part of application No. 11/185,736, filed on Jul. 21, 2005.

(51) Int. Cl. A61M 5/30

(52) U.S. Cl. 604/72

(57) ABSTRACT

A vial filling system for a needle-less injector, the system including an adaptor. The adaptor has a housing including a top and a side wall extending downwardly from the top. A recess is disposed in the side wall of the adaptor for receiving a vial. The vial has an inner cavity for receiving a liquid formulation. An inlet opening is located in the top of the adaptor, wherein when the vial is inserted into the recess the inlet opening is aligned with a nozzle of the vial. A needle extends from the top of the housing and in communication with the inlet opening. The needle is constructed and arranged to pierce a container of liquid formulation. A plunger is movably disposed within the cavity of vial for drawing the liquid formulation from the container into the cavity of the vial.

### RELATED APPLICATIONS

The present application is a continuation-in-part of U.S. patent application Ser. No. 11/121,439, entitled “Needle-less Injector,” filed May 30, 2002 and is related to U.S. patent application Ser. No. 11/185,736, entitled “Needless Injector and Ampule System,” filed Jul. 21, 2005.

### BACKGROUND OF THE INVENTION

1. Field of the Invention

The present invention relates to a vial filling system and method for filling a vial of a needle-less injector used for the subcutaneous delivery of a dose of liquid formulation to a human or animal, and more particularly, to a filling adaptor for a vial of a needle-less injection device that delivers a high-pressure jet of fluid through the epidermis of the human or animal.

2. Description of Related Art

The advantage of needle-less injection devices has been recognized for some time. Some of these advantages include: the absence of a needle stick injuries that presents a hazard to healthcare workers; the risk of cross-contamination between humans or animals is reduced; the risk of needle breakage in the tissue of the human or animal is eliminated; and the jet of liquid medicament is generally smaller than the diameter of a hypodermic needle and thus is less painful than a hypodermic needle.

Because of the well-known advantages of needle-less injection, there are many pneumatic or gas actuated needle-less injection devices that are designed to provide multiple doses to patients or animals. Most known needle-less injection devices operate by using a piston to drive the fluid to be delivered though a fine nozzle that creates a small, high pressure stream that penetrates the skin simply due to the high pressure. Multi-dose devices depend on a source of air or working fluid that is used to operate the piston that drives the fluid through the nozzle. Thus, a serious limitation of these devices is that they must have a readily available source of air or other fluid to drive the piston. This makes these devices impractical for use in the hospitals or clinics, and the field, especially in remote areas.

Because of the disadvantages of injection devices that use high-pressure fluids to drive the piston, a great deal of attention has been given to the development of a spring-powered needle-less injection device. The success of the known devices has been limited, due to problems associated with safety and reliability. The issues regarding safety generally involve the possibility of accidental discharge of the device. And the problems of reliability generally involve the device's ability to deliver a full, known dose of the liquid being delivered into the animal or human.

Safety issues generally arise in association with devices that have exposed triggers or include a ram or piston driving device that can extend beyond the inner housing of the injector. The risk of using this type of device is similar to the risks associated with the triggers on firearms, and that is the inadvertent pressing of the trigger, can result in the accidental or premature firing of the device.

Reliability issues include a broad spectrum of problems. One significant problem is the creation of a suitable jet or stream of fluid and the introduction of this jet on to the skin of the animal or human. Preferably, the jet will be a very fine jet that will impact a section of taut skin at an angle of incidence of preferably 90 degrees. Most of the energy of the stream is used to penetrate the skin when the jet impacts at approximately 90 degrees to the skin. Additionally, by keeping the skin taut prior to delivering the jet of fluid, the skin is not allowed to flex, and thus more of the energy from the jet is used to penetrate the skin rather than deflecting or moving the skin.

There are also significant disadvantages related to the containment of the fluid formulations in needle-less injectors. The reservoir of the injector can include an amount of liquid formulation ample to deliver numerous injections. Individual doses of a liquid formulation can also be delivered via the injector. The individual doses can be provided in a plurality of reservoirs coupled to the delivery device. A disadvantage of these types of liquid formulation delivery is that the user must have a large supply of liquid formulation on hand for treating numerous patients or animals. This decreases the practicality and use of the injectors in numerous environments.

Providing the desired amount of liquid formulation in a pre-dosed disposable vial helps solve the above-mentioned problems. However, a need still exists for allowing these pre-dosed vials to be filled in the field.

### SUMMARY OF THE INVENTION

According to one aspect of the present invention there is provided a hand-held, spring-powered, needle-less injector device that can deliver a dose of liquid, such as a medicament, both safely and reliably without an external power source.

In another aspect, the needle-less injector of the present invent prevents accidental discharge. The needle-less injector device has a trigger stop that prevents operation of the trigger when the inner housing in not in the firing position. An example of this trigger stop includes a protrusion that extends from the outer housing and impedes the movement of the trigger when inner housing is not in the firing position. The protrusion then moves away from the trigger when the inner housing is moved into the firing position.

In yet another aspect, the needle-less injector device of the present invention uses a single-use, disposable needle free vial containing a liquid for delivery. The vial includes a connector at one end and a nozzle and skin tensioner at the other end. The connector can be a bayonet type connector. The skin tensioner can be a ridge that surrounds the nozzle. The vial is easily insertable into the injector and provides for a safer healthcare environment.

It is still another aspect of the present invention to provide a field filling device that is attachable to the vial to enable the vial to be filled with a liquid from a bulk container to enable a vial to be filled with the desired dosage in the field.

Prior to injection, the user will attach a field filling adaptor to an empty vial. The adaptor is insertable into a bulk container of liquid formation. A plunger located in the vial draws the liquid into the vial until it is filled with the proper dosage amount. Thereafter the vial is removed from the adaptor and is ready to be inserted into the injector.

During operation of the injector, the user will position the ram at the cocked position and insert the vial into the leading end of the inner housing. The vial can be pre-filled with the liquid that is to be delivered to the animal or human as described above. Then the user presses the nozzle and skin tensioner against the animal or human, causing the inner housing of the device to move against the skin tensioning spring, into or relative to the outer housing to the firing position. Once the inner housing is moved to the firing position, the pressure of the skin tensioning spring is reacted against the animal or human, causing the skin to be stretched taut across the skin tensioner. This stretching of the skin across the skin tensioner will position the target area of the skin at a right angle to the vial and the nozzle. The movement of the inner housing to the firing position also results in the movement of a protrusion relative to the inner housing such that the protrusion no longer obstructs the movement of the trigger. The user then simply presses the trigger, which releases the ram, which in turn drives the fluid through the nozzle of the vial and into the animal or human's skin.

The ram may drive a separate plunger with a seal through the vial to expel the fluid in the vial through the nozzle of the vial. However, the ram may incorporate portions, or all, of the plunger. It is preferred that the ram will drive a separate plunger and seal will be used since this will enable the design of a one-time use plunger and seal.

Still further, it is contemplated that the use of a separate plunger will allow the use of a mechanical cocking device that will push against the ram to move the ram from an unloaded position to the cocked position.

According to these and other aspects there is provided a vial filling system for device for a needle-less injector including an adaptor. The adaptor has a housing including a top and a side wall extending downwardly from the top. A recess is disposed in the side wall of the adaptor for receiving a vial. An inlet opening is located in the top of the adaptor. When the vial is inserted into the recess the inlet opening is aligned with a nozzle of the vial. A needle extends from the top of the housing and is in communication with the inlet opening of the adaptor. The needle is constructed and arranged to pierce a container of liquid formulation. A plunger is movably disposed within the vial for drawing the liquid formulation from the container into the vial.

According to these and other aspects there is provided a filling adaptor for a vial of a needless injector. The adaptor includes a housing having a top and a side wall extending downwardly from the top. A recess is disposed in the side wall of the adaptor for receiving a vial. An inlet opening is located in the top of the adaptor, wherein when the vial is inserted into the recess the inlet opening is aligned with a nozzle of the vial. A needle extends from the top of the housing in communication with the inlet opening and the nozzle, wherein the needle is constructed and arranged to pierce a container of liquid formulation.

According to these and other aspects there is provided a method of filling a vial of a needless-injector device comprising the steps of providing a vial. The vial has opposed ends, one of the ends including a nozzle for receiving and ejecting liquid. A plunger is positioned within the other end of the vial. An adaptor is provided. The adaptor includes a recess for receiving the nozzle end of the vial and an inlet opening. A needle is in communication with the inlet opening. The adaptor is positioned on the nozzle end of the vial, wherein when the adaptor is positioned on the vial the nozzle, inlet opening and needle are in fluid communication. A source of liquid is pierced with the needle. The plunger is moved within the vial to draw the liquid from the source through the nozzle and into the vial. Thereafter, the adaptor is removed from the vial.

These and other objects, features, aspects, and advantages of the present invention will become more apparent from the following detailed description of the preferred embodiment relative to the accompanied drawings, in which:

### BRIEF DESCRIPTION OF THE DRAWINGS

FIG. 1 is a partial cutaway of the needless injector device of the present invention.

FIG. 2 is a top view of the device of FIG. 1.

FIG. 3 is an exploded view of the needless injector device of the present invention.

FIG. 4 is a cross-sectional view of the needless injector device shown in the ready position, prior to moving the inner housing into the firing position.

FIG. 5 is a cross-sectional view of the needless injector device of the present invention in the firing position.

FIG. 6A is a perspective view of an embodiment of the vial and seal of the present invention.

FIG. 6B is a top view of the vial and seal of FIG. 6A.

FIG. 7 is a perspective view of a carrying and cocking device for the needle-less injection device of the present invention.

FIG. 8 is a side view of the carrying and cocking device of FIG. 7.

FIG. 9 is a perspective view of the vial of the present invention.

FIG. 10 is a perspective view of the vial and field filling adaptor of the present invention.

FIG. 11 is a top view of FIG. 10.

FIG. 12 is a perspective view of the field filling adaptor.

FIG. 13 is a perspective cross-sectional view of the vial, seal and break-away plunger according to the present invention.

### DETAILED DESCRIPTION OF THE PREFERRED EMBODIMENTS

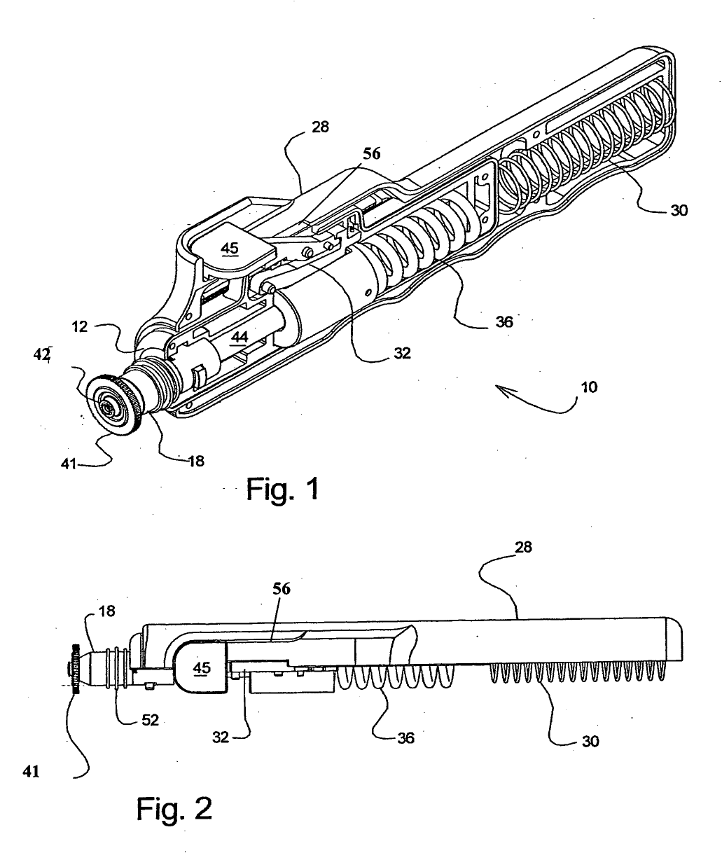

Referring to FIGS. 1-5, a hand-held, spring-powered, needle-less injector device 10 includes an inner housing 12 having a leading end 14, and a trailing end 16. The leading end 14 of the inner housing 12 is constructed and arranged to receive a vial 18 that is used to hold a fluid 20 that is to be delivered through the skin 22 covering the tissue of an animal or human 24 and into the tissue of the animal or human 24. It should be appreciated that although the present invention is described in relation to “skin” and “animal,” it is intended to include humans, animals and other surfaces.

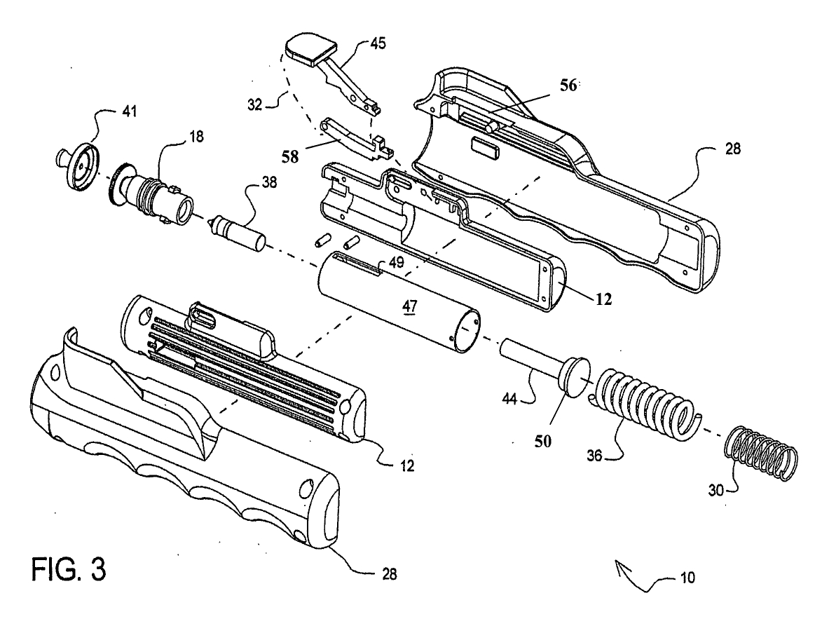

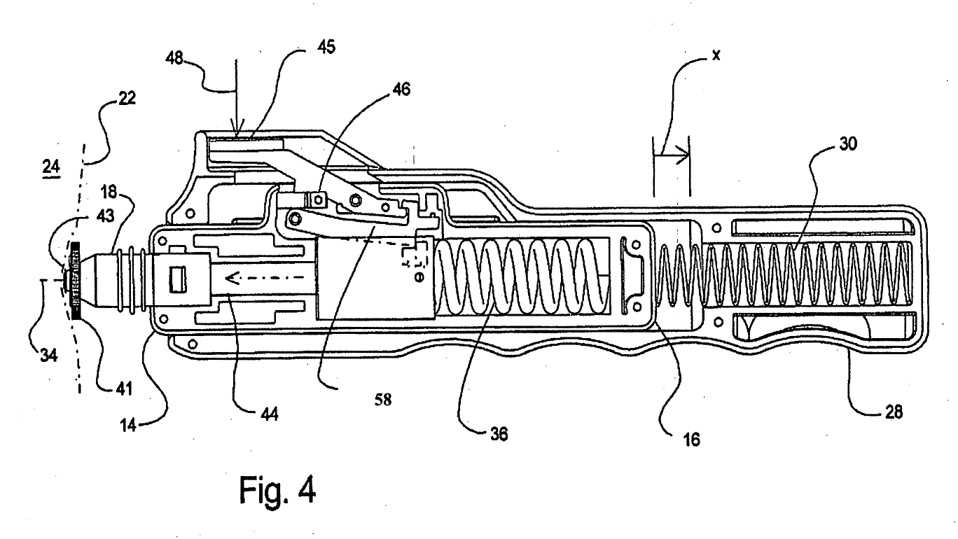

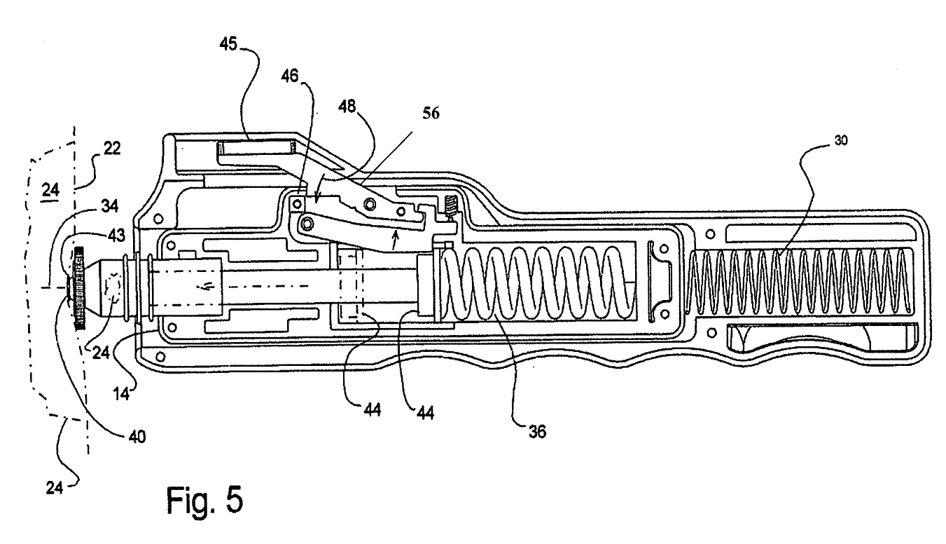

As illustrated in FIGS. 1 and 3, inner housing 12 is movably mounted within an outer housing 28 so as to slide along the axial direction thereof. The inner housing is movable from a ready position, illustrated in FIG. 4, to a firing position, illustrated in FIG. 5.

Inner housing 12 can be moved into the ready position of FIG. 4 by a skin tensioning spring 30 that is mounted between the inner housing 12 and the outer housing 28. The skin tensioning spring 30 has at least two primary functions. The first function of spring 30 is to cooperate with the structure of the vial 18 to pull the animal's skin 22 taut while positioning the skin 22 prior to delivering the fluid 20 into the animal or human 24. The second primary function of the skin tensioning spring 30 is to cooperate with a trigger mechanism 32 to ensure that the device 10 cannot be fired until the device 10 is properly positioned against the skin 22 covering the tissue of the animal or human 24, and the proper amount of pressure or force exists between the vial 18 and the skin 22.

The amount of pressure or force that is used to hold vial 18 against skin 22 is an important variable in the injection process. Needle-less injection devices are capable of delivering fluids through the skin 22 of the animal or human 24 by injecting a jet of fluid 34 into the skin 22 at a sufficiently high pressure and velocity so that fluid jet 34 penetrates through the skin 22 and into the tissue of the animal or human 24.

Important factors that contribute to the device's ability to accomplish the task of forming a jet of fluid 34 are the amount of energy that can be quickly and efficiently transferred to the fluid jet 34, and the device's ability to position the fluid jet 34 such that the energy of the jet is efficiently used to penetrate the tissue.

The energy to be transferred to fluid 20 is stored in an injection delivery spring 36 that drives a plunger and seal 38 into the vial 18 in order to force the fluid 20 through a nozzle 40 that forms the jet of fluid 34. Injection delivery spring 36 is positioned between a head 50 (FIG. 3) of a ram 44 and the trailing end of inner housing 12.

In order to obtain the most efficient delivery of the jet of fluid 34 into the skin 22 the nozzle 40 should be positioned at a right angle relative to the skin 22 as the jet of fluid 34 is delivered 22. Although the device may still operate at other angles, delivering the jet of fluid 34 at some angle other than a right angle could result in a component of the force with which the jet of fluid strikes the skin could be parallel to the skin rather than into the skin 22.

As illustrated in FIGS. 1 and 4-6B, vial 18 can include a skin tensioner 42 that surrounds nozzle 40. Skin tensioner 42 can be a disc 43 positioned approximately about the nozzle exit. It should be appreciated that skin tensioner 42 can take other shapes.

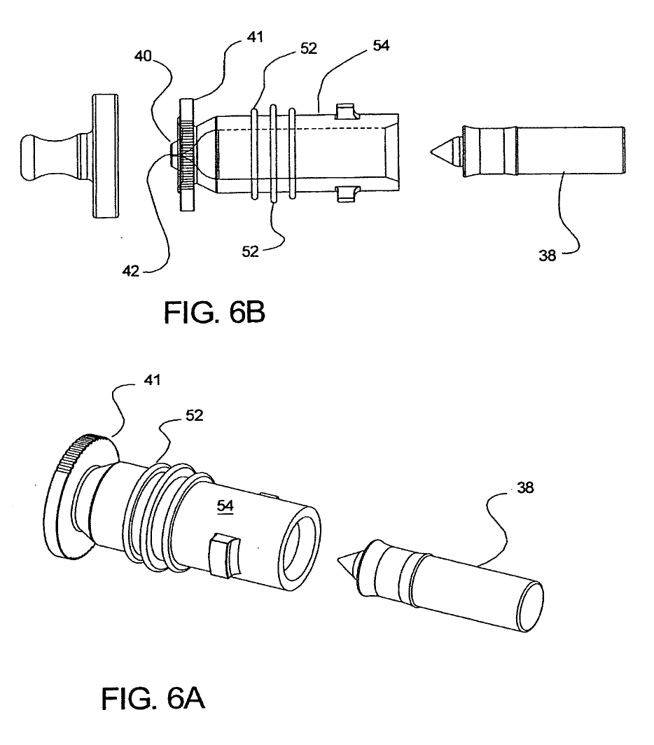

As shown in FIGS. 6A-6B, an installation ring 41 is provided on vial 18. The installation ring 41 aids the user in the insertion of vial 18 into the device 10 and in positioning the device 10 at a right angle to the skin as the jet of fluid 34 is to be delivered. The skin tensioner 42 may cooperate with the installation ring 41 to pull the skin taut as the device is pressed against the skin prior to delivery of the fluid jet 34. It should be appreciated that a certain minimum amount of force must be applied against the skin in order to ensure that the skin is drawn tight prior to the release of the jet of fluid 34.

The amount of force required to be applied against the skin varies depending on the physical characteristics of the patient being injected with the device 10. For example, an older human may require higher force to hold the skin taut as compared to a young person, simply due to the effects of aging on the elasticity of the skin. Accordingly, it is contemplated that the disclosed invention can be manufactured with different skin-tensioning springs, each skin tensioning spring being of a stiffness that is appropriate for a particular application. It is contemplated that the force imposed by the skin tensioning spring may be made adjustable, for example by adding a threaded plug that screws against the spring to add pre-tension. However, it is preferred that the force imposed by the skin tensioning spring should not be adjustable or replaceable by the end user, but is preferably pre-calibrated during assembly. The outer housing 28 and a cocking and storage mechanism for use with the device 10 will be color coded to inform the user of the pre-set skin-tensioning force for that particular injector device 10.

Thus, in operation the user selects an injection device with the appropriate skin pre-tension spring 30 and injection delivery spring 36, and selects a vial 18 that will contain a desired fluid to be delivered into the tissue of the animal. The vial 18 will be attached to the leading end 14 of the inner housing 12, preferably through the use of a bayonet-type connector, and mated to a seal 38 that may be a part of the plunger and seal 38. The plunger 38 is driven through the vial 18 by spring powered ram 44 that is movable from a safe, cocked position, illustrated in FIG. 4, to an unloaded position, illustrated by dashed lines in FIG. 5. As shown in FIG. 3, the spring powered ram 44 rides within a sleeve 47 that includes a slot 49 for accepting latching components of the spring mechanism 32.

The variation of the skin pre-tension spring 30 and injection delivery spring 36 allows the needle-less injector device 10 to be tailored for a particular application. For example, a needle-less injector device 10 for use on a child would have one particular combination of skin pre-tension spring 30 and injection delivery spring 36, while the combination of skin pre-tension spring 30 and injection delivery spring 36 for an adult male would likely be a different combination. Accordingly, the disclosed invention can the adapted for use on a variety of animals or humans, and for the delivery of a variety of types injections or depth of delivery of the fluid by varying the skin pre-tension spring 30 and injection delivery spring 36.

Referring once again to FIGS. 1-5, outer housing 28 includes an aperture 56. A trigger 45 is mounted in inner housing 12 and protrudes through aperture 56 so as to be engageable by a user. Trigger mechanism 32 includes a link 58 (FIG. 3) that controls the release of ram 44. As can be understood from comparing FIGS. 4 and 5, the firing of the device 10 to deliver a dose of fluid is accomplished by pressing the trigger 45 after the device 10 is in the firing position, illustrated in FIG. 5. However, the trigger 45 of the trigger mechanism 32 can only release the plunger and seal 38 when the device 10 is in the firing position, illustrated in FIG. 5. When the device 10 is in another position (other than the firing position), such as the ready position, the trigger link 58 of mechanism 32 cannot be pressed to release the ram 44. The release of the ram 44 is prevented for safety and for efficacy of the injection.

As illustrated in FIGS. 4 and 5, unwanted activation of the trigger mechanism 32 is accomplished by positioning a protrusion 46 below trigger 45. The protrusion 46 prevents movement of the trigger 45 in the direction of arrow 48, preventing the release of ram 44, and thus preventing the firing of the device 10. According to a preferred embodiment of the invention the protrusion 46 extends from the outer housing 28 to a location under the trigger 45. The protrusion 46 is positioned such that it interferes with the movement of the trigger 45 until the device 10 is in the firing position, as illustrated in FIG. 5.

In the preferred example of the invention, the movement of the inner housing 12 relative to the outer housing 28 moves the position of the trigger 45 (which is mounted from the inner housing 12) relative to the outer housing 28, which holds the protrusion 46. The amount of movement of the outer housing 28 relative to the inner housing 12 is accomplished against the force of the skin-tensioning spring 30.

The stiffness of the skin-tensioning spring 30 is selected such that the appropriate amount of force is imposed against the skin 22 of the animal or human 24. The stiffness of the skin-tensioning spring 30 is calculated from the well-known formula:
F=k*x,

where F is the required force at the firing position, x is the distance of travel of the inner housing 12 relative to the outer housing 28 to position the device in the firing position (where the protrusion 46 does not impede movement of the trigger mechanism 32), and k is the spring constant of the skin-tension spring 30.

Once the inner housing 12 is positioned relative to the outer housing 28 such that the desired amount of skin tensioning force is applied to the skin 22 against the vial 18, which also positions the device in the firing position, the pressing of the trigger 45 causes the release of the spring powered ram 44 from the cocked position only when the inner housing is in the firing position.

As shown in FIGS. 6A and 6B, vial 18 will generate fluid jet 34 through nozzle 40. Vial 18 includes a plurality circumferential stiffening ribs 52 that extend around a body 54 of vial 18. These stiffening ribs help reduce the amount of deflection of the body 54 of the vial 18 during the delivery of an injection.

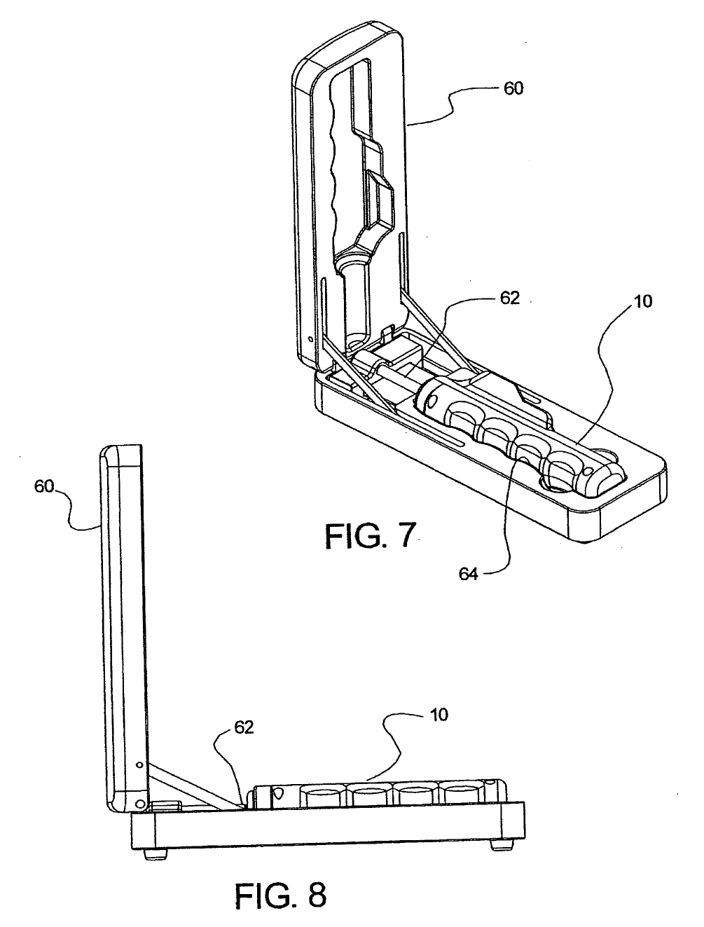

Referring to FIGS. 7 and 8, it should be understood that the disclosed needle-less injection device can be used with a combined cocking and carrying device 60. The cocking and carrying device includes a cocking ram 62 that is used to push the spring powered ram 44 back to the “ready” position shown in FIG. 4. The cocking and carrying device 60 also includes a cradle 64 that retains the outer housing 28 while the cocking ram 62 is pushed against the spring powered ram 44.

Cocking ram 62, when pushed against spring powered ram 44, moves the ram into the “ready” position illustrated in FIG. 4. It should be understood that the cocking and carrying device 60 will cock the needle-less injection device 10 once the device is positioned in the cradle 64 and the cocking and carrying device 60 is closed. Thus, device 60 will serve as both a cocking device and case for transporting and storing the needle-less injection device 10. However, prior to placing device 10 within the cocking and carrying device, the user must first insert vial 18 into leading end 14 of inner housing 12. As discussed above, disposable vial 18 can contain a dose of liquid formulation for delivery. Vial 18 can be made of a readily injection moldable material, such as a pharmaceutical grade polypropylene or a polymer material. One example of such a polymer material is TOPAS®, manufactured by Ticona Engineering Polymers, A division of Celanese.

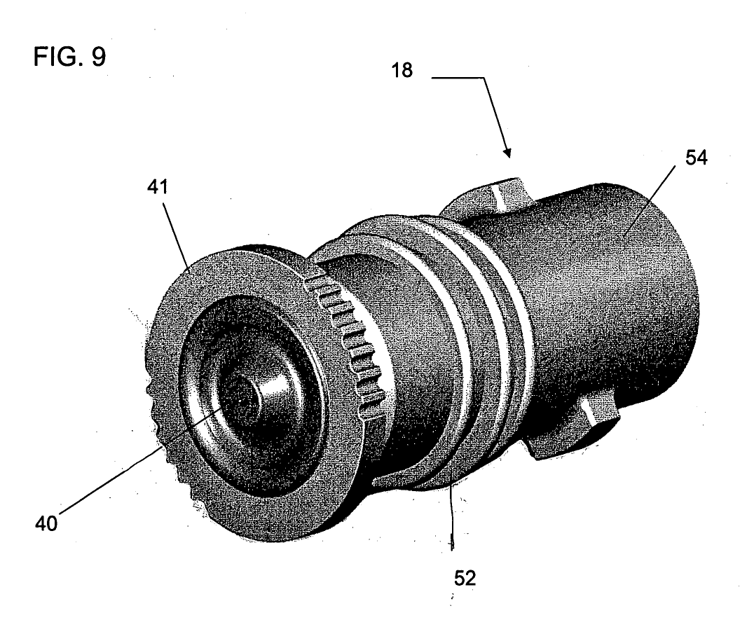

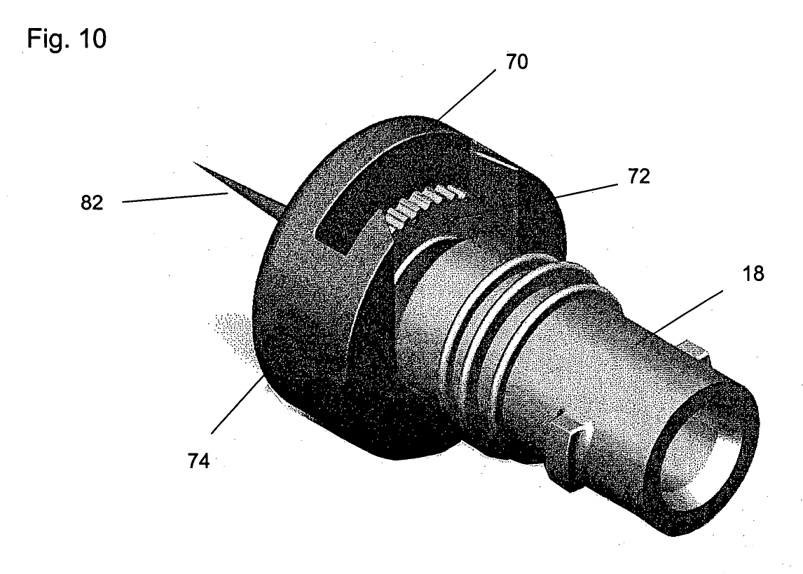

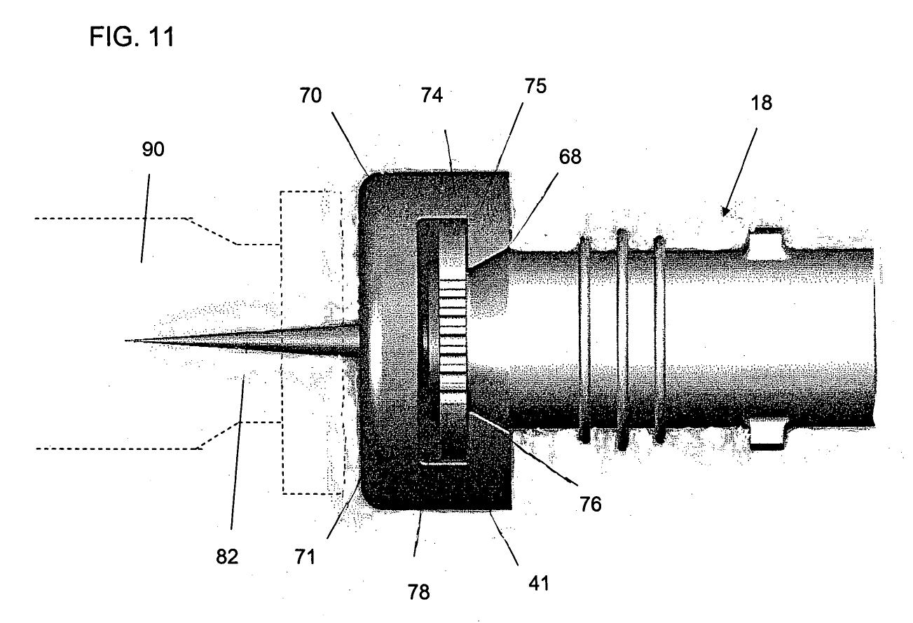

Referring to FIGS. 9-12, the field-filling adaptor system of the present invention will be described. In order to fill vial(s) 18 from a larger bulk container 90 (FIG. 11) of vaccine or other liquid medicine, the present invention contemplates the use of an adaptor 70 that can be removably attached to the vial. Adaptor 70 is similar in shape to a cap and includes a top 71 and a sidewall 74. Sidewall 74 includes a lip 75 extending inwardly from a bottom thereof. A recess 72 is located in lip 75 of sidewall 74 and defined by angled edges 76. The adaptor is inserted on the nozzle end of vial 18 by way of recess 72. When assembled onto vial 18 a shoulder 68 of vial 18 engages angled ends 76 of wall 74 to secure the adaptor on the vial. As shown in FIGS. 10 and 11, installation ring 41 of the vial is located within an aperture 78 of adaptor 70 when assembled.

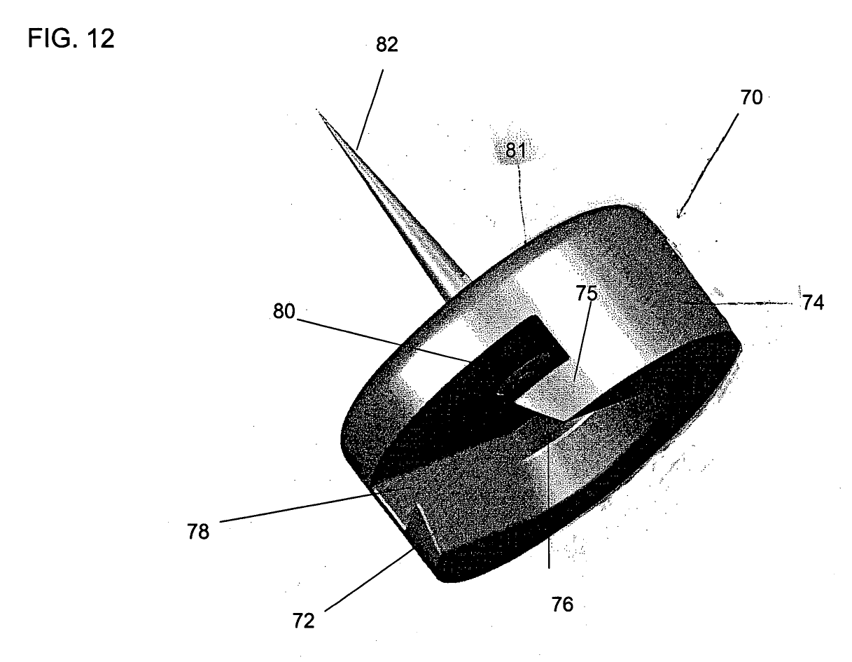

As shown in FIG. 12, adaptor 70 includes an inlet opening 80 that communicates with a needle 82 formed on the top of the adaptor. Inlet 80 is in fluid communication with nozzle 40 when the adaptor is placed on vial 18. The adaptor can be made of a plastic material, such as a polyethylene, polycarbonate or other material. The adaptor can be injection molded in a single piece or manufactured in another manner.

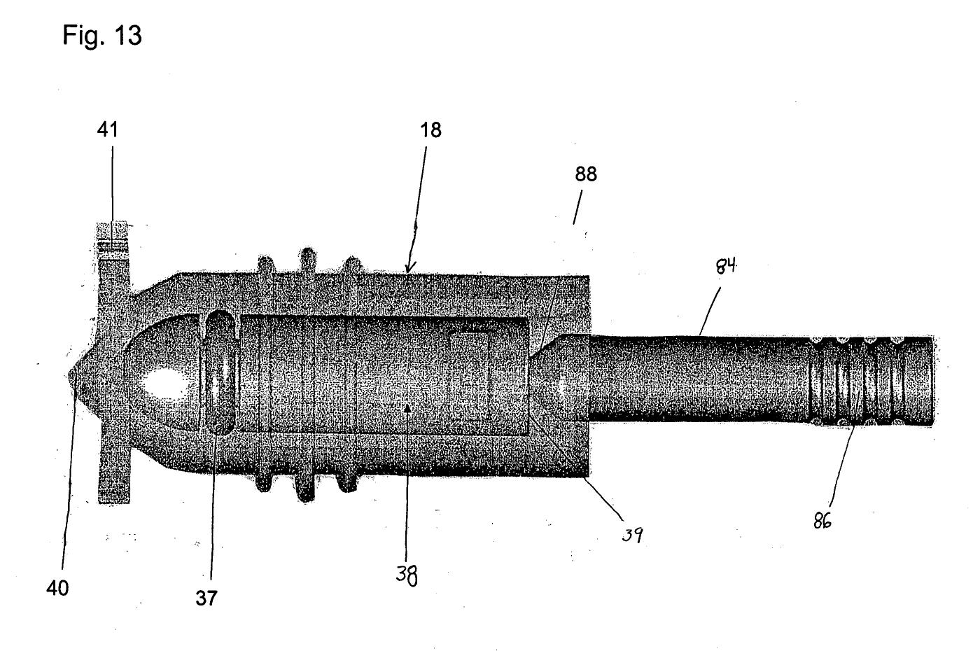

As discussed supra, vial seal/plunger 38 is movably located within vial 18 to force the dose of medicament through the nozzle. Referring to FIG. 13, vial plunger 38 is movably located within vial 18 and can include a handle 84 attached to end 39 of the plunger. Handle 18 includes a grip area 86 located at one end and an area of reduced diameter 88 at its other end.

Plunger 38 includes a seal 37 located at its end. Seal 37 can be an o-ring or similar sealing device. Seal 37 creates a seal between plunger 38 and the inner wall of vial 18.

During operation, the user inserts the plunger 38 into vial 18 and moves the same within vial 18 using handle 84 until plunger 38 reaches the position shown in FIG. 13. Next, the user slides adaptor 70 onto installation ring 41 of the vial so that nozzle 40 and inlet opening 80 of the adaptor are aligned. Next, as shown in FIG. 11, the user pierces the seal of the bulk container 90, shown by dashed line, such that the tip of needle 82 is received within the liquid medicament contained therein. Handle 84 can then be pulled backwards and due to seal 37, the liquid medicament is drawn from the container through needle 82 and nozzle 40 into vial 18. Once the desired dose has been drawn into vial 18, handle 84 can be snapped from plunger 38 at the area of reduced diameter 88 and disposed.

The injector is placed into the cocking device and the lid is closed to “cock” the injector. The cocking device is also used as a storage compartment for the injector when not in use. Once the injector is “cocked”, the user will insert the pre-filled vial and seal into the opening in the end of the injector. The vial is designed to purge a small amount of liquid at the time of insertion so as to maintain the proper quantity of liquid for the injection. The vial will snap into place as it is rotated in the injector housing. The injector is now ready to use.

As described above, the user will press the face of the vial against the skin and depress the trigger to give the injection. The injector inner housing slides inside the outer housings, which creates an interlock so that the device cannot be operated until the proper tension against the skin is established. When the trigger is pressed, the trigger latch will release the hammer and the hammer will move the vial seal into the vial. The main pressure spring will deliver enough pressure to allow the liquid to pierce the skin.

After the injection has taken place, the vial is removed with the seal and discarded. The injector is then placed into the cocking mechanism and reloaded for the next injection.

The vial and seal assembly can be pre-filled or field filled with the use of an adapter and a break-away plunger. The current design is for a fixed dosage of 0.5 cc. When using the field filling device, the user will attach the field filling device to the vial, insert the break-away plunger into the vial, place the adapter into the bulk container and draw the liquid into the vial by pulling on the break-away plunger. When the vial is filled to the proper level, the user will snap the handle off of the break-away plunger, remove the vial from the adapter, and insert the vial into the injector housing. After the injection is complete, the user will remove the vial from the injector and discard. The plunger will be recessed into the vial and cannot be reused.

Plunger 38 and handle 84 can be made of a single piece of polycarbonate that has been injection molded or made by any other suitable manufacturing technique to allow the handle and plunger to be separated after the vial has been filled. Handle 84 enables the user to download the liquid to a desired level. Vial 18 can be formed of a clear material and could include indicia to aid the user. Moreover, the length of the plunger and handle can be varied to further control filling.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is claimed is:

1. A vial filling system for a needle-less injector, the system comprising:
an adaptor, the adaptor having a housing including a top and a side wall extending downwardly from the top;
a recess disposed in the side wall of the adaptor for receiving a vial, the vial having an inner cavity;
an inlet opening located in the top of the adaptor, wherein when the vial is inserted into the recess the inlet opening is aligned with a nozzle of the vial;
a needle extending from the top of the housing in communication with the inlet opening, wherein the needle is constructed and arranged to pierce a container of liquid formulation; and
a plunger movably disposed within the cavity of vial for drawing the liquid formulation from the container into the cavity.

2. The filling system of claim 1, wherein the plunger has opposed ends, and further comprising a handle removably attached to one of the ends of the plunger.

3. The filling system of claim 1, wherein the adaptor includes an aperture in communication with the inlet opening.

4. The filling system of claim 3, wherein the vial has an installation ring located at one end thereof about the nozzle of the device, the installation ring being located within the aperture when the adaptor is positioned on the vial.

5. The filling system of claim 1, wherein the vial, adaptor and plunger are made of a plastic material.

6. The filling system of claim 5, wherein the vial is an injection molded polymer.

7. The filling system of claim 5, wherein the plunger is made of a polycarbonate material.

8. The filling system of claim 1, wherein the sidewall includes a lip extending inwardly from a bottom thereof.

9. The filling system of claim 8, wherein the lip includes an angled edge at each end to define the recess, the angled edges being constructed and arranged to mate with a shoulder of the vial to secure the adaptor on the vial.

10. A filling adaptor for a vial of a needless injector, the adaptor comprising:
a housing including a top and a side wall extending downwardly from the top;
a recess disposed in the side wall of the adaptor for receiving a vial;
an inlet opening located in the top of the adaptor, wherein when the vial is inserted into the recess the inlet opening is aligned with a nozzle of the vial; and
a needle extending from the top of the housing in communication with the inlet opening and the nozzle, wherein the needle is constructed and arranged to pierce a container of liquid formulation.

11. The adaptor of claim 10, wherein the adaptor is made of a plastic material.

12. The adaptor of claim 11, wherein the sidewall includes a lip extending inwardly from a bottom thereof.

13. The adaptor of claim 12, wherein the lip includes an angled edge at each end to define the recess, the angled edges being constructed and arranged to mate with a shoulder of the vial to secure the adaptor on the vial.

14. A method of filling a vial of a needless-injector device comprising the steps of:
providing a vial, the vial having opposed ends, one of the ends including a nozzle for receiving and ejecting liquid;
positioning a plunger within the other end of the vial;
providing an adaptor, the adaptor including a recess for receiving the nozzle end of the vial, an inlet opening and a needle in communication with the inlet opening;
positioning the adaptor on the nozzle end of the vial, wherein when the adaptor is positioned on the vial the nozzle, inlet opening and needle are in fluid communication;
piercing a source of liquid with the needle;
moving the plunger within the vial to draw the liquid from the source through the nozzle and into the vial; and
removing the adaptor from the vial.

15. The method of claim 14, wherein the plunger includes a handle removably attached at one end thereof and further comprising the step of snapping the handle off of the plunger after the vial has been filled with the liquid.
